(19) United States
(12) Patent Application Publication
(10) Pub. No.: US 2009/0208593 A1
(43) Pub. Date: Aug. 20, 2009

(54) PHYTO AROMA MICRO-NUTRITHERAPEUTIC COMPOSITIONS IN ORODIETOLOGY

(76) Inventor: Gaetano ZANNINI, Biot (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 12/033,515

(22) Filed: Feb. 19, 2008

(51) Int. Cl. A61K 36/00; A61P 3/00; A61P 1/00

(52) U.S. Cl. 424/725

(57) ABSTRACT

The invention concerns a composition comprising at least a nutrient and at least a phytotherapeutic compound. The composition according to the invention is characterized in that it also comprises at least an essential oil and/or essence and it is in the form of a granule. The invention is used as a complement of a varied and balanced diet for indications such as aging, weight loss, discomfort related to hormonal changes of the pre-menopause and menopause, drainage, digestion, intestinal transit, relaxation, calm, microcirculation, appeasement of the bacterial flora, painful joints, physical and mental fatigue, protection of the immune system, or even whiten teeth and reduce urge to smoke.

The invention relates to compositions combining the benefits of phytotherapy, aromatherapy and micro-nutrition, and their manufacturing processes. The compositions according to the invention comprise at least: a nutrient, a phytotherapeutic compound and an essential oil.

Phytotherapy, aromatherapy and micro-nutrition are well-known unconventional medicines, and are often used to supplement therapeutic treatments.

Phytotherapy, etymologically treatment by plants, is a therapeutic method that uses the action of medicinal plants.

In phytotherapy, plants may be used fresh (rarely), or dried. The plants or plant parts enter various preparations in order to preserve their active ingredients and avoid denaturation. They are used as plant powders, liquid extracts, dried extracts. Generally, ground plants are macerated in water or in low alcoholic water/alcohol mixture. The liquid extract is then evaporated, we get as a first step a soft extract, which in turn is sprayed on an inert support until production of a powder: the dried extract. This powder can then be administered in the form of tablets, capsules, or sachets.

Aromatherapy is the therapeutic use of essential oils and essences. This differs from phytotherapy that makes use of all the components of the plant.

The term aromatherapy covers very different medical practices using essential oils in the form of unction, dissolution in an oil, cream or lotion, oil emulsion in water, for external use. Aerosols obtained by nebulization of essential oils are also used. The use of rectal suppository is very useful for certain therapeutic applications.

Phytotherapy and aromatherapy are known to be used in the treatment of diseases that characterize today's society, such as stress, insomnia, depression, obesity, allergies, acne, cellulite, weight problems, cholesterol, hypertension or diabetes.

Micro-nutrition is the use of medical foods and micronutrients by the cell.

The action of proteins, carbohydrates, lipids, anti free radicals, anti-oxidants, vitamins, minerals, and other fibers in bio-available form, particularly by oral route, was studied with the aim of improving by micro-nutrition the systems of the body and all the functions of human beings, to optimize the performance, such as energy, intellectual capacity, the immune defences and to extend the maintenance in a state of good health and to feel better. The dietary imbalances and deficiencies in essential cellular nutrients are at the beginning of a cellular oxidative stress which can lead to degenerative diseases. Indeed, the incorporated micronutrient intakes by the body from our daily diet are not always sufficient and are often less than the recommended dietary intake. Our body is composed of cells that are to receive a daily accurate and balanced quantity of nutrients essential to their functioning. These nutrients are extracted from the diet by digestion. However, nourishment for most of people provide only 25% of cellular essential nutrients. Nutritherapy is therefore used to correct these deficits notably in fibers, anti-oxidants, anti free radicals, vitamins, minerals by administering to the cells nutritional supplements adapted to the different nutritional deficiency situations. For example, it has been shown from SU.VI.MAX clinical trial that such a cellular micro-nutrition, with vitamins, minerals, anti-oxidants, has a beneficial action on mortality and reduction of cancer of men. Moreover, it is also known that a daily moderate dose of nutritional supplement of vitamins and minerals combined to an equilibrate diet is good for the body.

However, it is much more common that these three types of therapies are used independently of each other.

There are also compositions combining notably phytotherapy and aromatherapy. We find particularly formulations such as soft capsules or pills that contain an active extract of plant, phytotherapeutic compound, and an essential oil in liquid form. The essential oil, which can still be found in the liquid state, leads to stability problems with such formulations. In addition, there is a high risk of toxicity with some liquid essential oil preparations used in high and uncontrolled doses. The essential oils are also particularly volatile, and it is really difficult to encapsulate it in dry form.

Furthermore, from our knowledge, there is no single form of therapy combining the three, namely an association between a phytotherapeutic compound, an essential oil and a nutrient, with the exception of water. We can find formulations combining only two of the three therapies, particularly in the form of capsules or soft capsules containing the oil in liquid form which leads to problems of stability.

Moreover, in order to associate the benefits of the three therapies, it is often necessary for the patient to take at least two separate treatments, mostly three, one for each therapy. In addition, these formulations, depending on their respective dosage, should be taken one to three times per day. This generally leads inevitably to reduce the observance of such treatments, and thus to reduce their beneficial effects.

Thus, such formulations of the prior art have numerous disadvantages.

Considering the above, a problem to be solved by the invention is to realize a composition of a nutrient and a phytotherapeutic compound, stable and nontoxic composition bringing the benefits of phytotherapy, aromatherapy and nutritherapy in a single form without presenting the disadvantages of the formulations according to the prior art.

The solution of the invention to this problem concerns, according to a first object, a composition as above, further comprising an essential oil and/or essence, and is in the form of a granule.

It concerns, according to a second object, a process for preparing a composition in the form of a granule as defined above, comprising the following steps:
granulation in the presence of a crystalline solid powdery phase comprising a nutrient and a phytotherapeutic compound and a liquid phase, which comprises a gum and an essential oil and/or essence;
coating by spraying of the liquid phase and sprinkling of solid crystalline powdery phase;
drying the coated granules obtained; and
screening for obtaining calibrated granules.

Thus, the composition according to the present invention presents the following advantages:
it combines the benefits of phytotherapy, aromatherapy and micro-nutrition in only one formulation;
it incorporates the essential oil in dry form in a granule;
it is more stable and less toxic than the formulations incorporating the essential oil in liquid form;
it promotes observance to treatment by the patient;
it is easy to use by the patient by its orally soluble formulation granule.

Advantageously:—the nutrient is a protein, a carbohydrate, a lipid, a fiber, an antioxidant, an anti free radical, a vitamin, a mineral salt, a trace element, or a mixture of those;—the nutrient concentration is comprised between approximately 1 and approximately 50% of the total weight of the composition;—the phytotherapeutic compound is an active plant extract of a whole plant or of a plant part;—the phytotherapeutic compound concentration is comprised between approximately 1 and approximately 80% of the total weight of the composition;—the essential oil and/or essence, taken alone or in combination, is obtained by distillation drive to water vapor of an aromatic plant or by pressing and scraping citrus fruit zests;—the concentration of essential oil and/or essence is comprised between approximately 0 and approximately 50% of the total weight of the composition;—the granule is orally soluble;—the composition comprises at least lycopene, tannin or polyphenol, an oleuropeoside, fucus, lettuce Sea and spirulina as a nutritional complex associated with at least a phytotherapeutic compound and at least an essential oil and/or essence in the form of orally soluble granule;—the process of preparation of a composition according to the invention incorporates all compounds in dry form;—the composition according to the invention comprising a nutrient, a phytotherapeutic compound and an essential oil and/or essence in a granule form, has a synergistic phytotherapeutic, aromatherapeutic and nutritherapeutic activity; and the composition according to the invention comprising a nutrient, a phytotherapeutic compound and an essential oil and/or essence in form of granule used as a complement of a varied and balanced diet for indications such as aging, weight loss, discomfort related to hormonal changes of the pre-menopause and menopause, drainage, digestion, intestinal transit, relaxation, calm, microcirculation, appeasement of the bacterial flora, painful joints, physical and mental fatigue, protection of the immune system, or even whiten teeth and reduce urge to smoke.

The invention will be better understood from the following non-limitative description.

Throughout this text, and unless otherwise specified, it is understood that when concentrations intervals are given, they include upper and lower bounds of that interval. Similarly, unless otherwise noted, the proportions of the various constituents of the composition are expressed in percentage by weight (w/w) of the total weight of the composition.

The invention relates to phyto aroma micronutritherapeutic compositions in Orodietology®.

Orodietology® uses in combination natural micro-nutrition to improve the cell response to phytotherapeutic and aromatherapeutic treatment.

According to the invention, the composition comprises at least a nutrient, at least a phytotherapeutic compound, and also at least an essential oil and/or essence.

Orodiethology® associates in the same solid form according to the invention natural micro-nutrition, phytotherapy and aromatherapy. The compositions according to the invention incorporate particularly Beta-Meditril®. Beta-Meditril® is a nutritional complex, which can revitalize the cell, comprising anti-oxidant and anti free radical compounds such as lycopene found in tomato, tannin or polyphenol found in grapes pips, blood expectorant such as oleuropeoside found in extract of olive tree, and micro-nutrients such as trace elements, minerals, vitamins, fibers, amino acids, essential fatty acids brought by the three algae fucus, lettuce sea and spirulina.

Anti-oxidants and anti free radicals are particularly used to improve the cell response. A cell in good condition can only be more reactive to a combined phytotherapeutic and aromatherapeutic treatment.

By nutrient, we understand any organic and mineral compound needed for the body to ensure its development and maintenance, with the exception of water.

The nutrient according to the invention can be chosen from energetic nutrients, such as proteins, carbohydrates, lipids, as well as non-energetic nutrients, such as fibers, antioxidants, anti free radicals, vitamins, mineral salts, trace elements, taken alone or in combination.

Preferably, the compositions according to the invention comprise at least lycopene, tannin or polyphenol, oleuropeoside, fucus, lettuce sea and spirulina as a nutritional complex called Beta-Meditril®.

In the compositions according to the invention, the concentration of nutrient is comprised between approximately 1 and approximately 50%, preferably between 2 and 40%, more preferably between 10 and 30% of the total weight of the composition.

By phytotherapeutic compound, we understand an active plant extract derived from a whole plant, parts of plants, such as leaf, bark, bud or root, or fruit, an extraction from a plant, taken alone or in combination.

As non-limitative examples of plants from which a phytotherapeutic compound can be extracted we can find medicinal plants included in the Pharmacopoeia, particularly in the traditional Pharmacopoeia.

Preferably, the phytotherapeutic compound is extracted from, as non-limitative examples, tomato, grapevine, olive tree, kiwi, cameline, acacia, yam, soya, sage, fennel, liquorice, coffee tree, pineapple, guarana, hibiscus, tea tree, ash, orthosiphon, cherry tree, corn, blueberry, ginkgo, cypress, tamarind tree, fig tree, plum tree, apple tree, heather, cranberries, passionflower, lime tree, lemon balm, lavender, orange tree, harpagophytum, horsetail, nettle, meadowsweet, hawthorn, basil, ginseng, ginger, eleutherocoque, pine, grapefruit, blackcurrant bush, marshmallow, kudzu, plantain, cacaotier, wheat, barley, oat, peppermint, camomile or shiitake and propolis.

In the compositions according to the invention, the concentration of phytotherapeutic compound is comprised between approximately 1 and approximately 80% of the total weight of the composition, preferably between 2 and 40%, more preferably between 10 and 30% of the total weight of the composition.

By essential oil, we understand a concentrated and hydrophobic liquid of volatile aromatic compounds from a fresh or dry plant. Essential oils are obtained either by distillation and extraction drive to water vapor in aircraft called distillers, by extraction by cold expression, or by extraction with supercritical CO2. Preferably, the essential oil is obtained by distillation drive to water vapor of an aromatic plant listed above.

By essence, we understand a complex set of volatiles odoriferous substances obtained by cold expression, that is to say by pressing and scraping of citrus fruit zests, by steam distillation of water, by rectification by hot enfleurage, by cold enfleurage, by solvent extraction, or by maceration. Preferably, the essence is obtained by pressing and scraping citrus fruit zests.

As non-limitative examples of citrus fruits, we can find the species of oranges, lemons and limes, mandarins, clementines, tangerines, and pomelos.

In the compositions according to the invention, the concentration of essential oil and/or essence is comprised between approximately 0 and approximately 50% of the total weight of the composition, preferably between 0 and 20%, more preferably between 0 and 5% of the total weight of the composition.

In an embodiment of the invention, the composition is in the form of a granule.

For the preparation of a composition according to the invention, we advantageously use the granulating process called Technabio™ disclosed in the patent document published under the number WO97/04861, which implements a granulator with a rotating turbine like coating turbine type.

For the preparation of a composition according to the invention, we more advantageously use the granulating process called Technabio™ II, which includes a dynamic stage drying, for example of around 15 minutes.

By granule, we understand a progressive, intimate and dynamic monoassociation, of at least a nutrient, at least a phytotherapeutic compound and at least an essential oil and/or essence.

According to an embodiment of the invention, the different compounds, preferably natural, are associated in spherical and dry form without any inert excipient, and preferably without external protective layer, to form a granule. There is successive aggregation in spherical shape around the micro-monoassociation of origin, of all other micro-monoassociations with the same characteristic and composition. The progressive union of micro-monoassociations between them is determined by a dynamic and uniform process of concentration from the center to the periphery. The diameter of the monoassociation progressively increases towards obtaining the desired dose report diameter, and therefore the correct dosage of the product. An active synergy exists between the active compounds of micro-monoassociations constituting the monoassociation or granule according to the invention. This synergy gives efficiency, speed, easiness and safety to use the granule. The micro-monoassociations being at the same time containing and contained, they are closely linked and protect each other.

Advantageously, according to the invention the granule is orally soluble. It gradually dissolves, releasing immediately and simultaneously the beneficial active compounds. The spherical shape is retained until the complete dissolution of the granule. This form of synergistic aggregation enables a unique bio availability for more efficiency and speed.

According to an embodiment of the invention, the absence of gelatin, inert excipients, alcohol, and conservatives in the granules, and advantageously, the absence of external protective layer, offers a great convenience and safety to the products.

Advantageously, orally soluble granules can be sucked so that the active compounds can be dissolved directly in the mouth and act immediately avoiding digestion enzymes and enterohepatic cycling.

For example, according to an embodiment of the invention, in a first step of the process according to the invention, we make a granulation in the presence of a solid powdery phase formed by the crystalline substance comprising at least a nutrient and at least a phytotherapeutic compound and a liquid phase comprising a gum and at least an essential oil and/or essence. The compounds of the liquid phase can be added one after the other. In quantitative terms, we can use for example, 100 grams of solid powdery phase for granulating, and 10 to 30 grams of liquid phase. The granulation is performed approximately at room temperature and in the presence of humidity below 70%.

The final dimensions of the desired granule are obtained by successive coating. These coatings are produced by spraying the liquid phase and then sprinkling above the solid crystallized powdery phase. A static drying is performed at the end of each coating stage. The drying temperature is between about 40° C. and about 55° C. and, preferably, between about 45° C. and about 50° C. The drying time is more than 30 minutes, for example about 1h30.

We can advantageously use Technabio™ II process. In this process, the drying is dynamic and quicker, from around 15 minutes to 30 minutes.

To obtain calibrated granules, we make a sieve. The mesh sieves are suitable for obtaining desired calibrated granules.

The granule may contain an external protective layer eventually coloured.

For the preparation of the external protective layer, we firstly prepare, a liquid or paste phase comprising liquid extracts of plants, essential oils, gums, water, alcohol. In quantitative terms, this liquid or paste phase may comprise between 1 and 20% by weight of aroma and between 5 and 40% by weight of alcohol, percentages given by weight of the total weight of the considered phase.

Then we spray this liquid or paste phase on the granule formed before. This phase ensures cohesion of the external protective layer to the granule. We sprinkle the all with the crystalline powdery phase. We then perform a final drying at temperatures between about 40° C. and about 55° C. and, preferably, between about 45° C. and about 50° C., for example in the range of 45° C. in order to obtain orally soluble granules according to the invention.

All the compounds are incorporated in dry form in the granules according to the invention. These granules are likely to be stocked and stored for a relatively long time, such as several months, without altering their qualities. The granules prepared according to the invention are dose prescribed so as to enable the patient to take 1 to 6 orogranules, preferably from 2 to 4 orogranules, per day by 2 shots, in addition to a varied and balanced diet.

The examples below show granules according to the invention.

For each composition according to the invention of the examples below, phytotherapeutic compounds can be incorporated in the solid powdery phase in the form of dry extracts and/or in the liquid phase in the form of fluid extracts. However, powders insoluble in aqueous phase are only incorporated in the solid powdery phase. Active compounds essentially available as a liquid or fluid such as essential oils, vegetable oils or extract of tomato, are only incorporated in the liquid phase.

The drying of granules obtained is done in oven for 1 hour to 2 hours depending on the composition, at temperatures between 45 and 50° C. Then a sieve with mesh from 4 mm in diameter to the desired diameter is realised. At the end, we obtain orally soluble granules calibrated from 200 to 500 mg of average weight.

### EXAMPLE 1

### Granule A40 ANTI AGE

We realize a granulation using the Technabio™ process from a solid powdery phase and a liquid phase: granulation solution. Beta-Meditril® is a nutritional complex comprising lycopene, tannin or polyphenol, oleuropeoside, fucus, lettuce sea and spirulina. In this example, tannin or polyphenol, spirulina, lettuce sea, grapes pips, olive tree extract, acacia fibers, selenium, zinc are incorporated in the solid phase; lycopene, oleuropeoside, fucus, kiwi oil, camelina oil, orange essential oil, lemon essential oil are incorporated in the liquid phase.

The percentages reflect the percentages of active ingredients in the orogranule finished:

| | |
|---|---|
| Beta-Meditril ® | 1 to 10% |
| Lycopene | 10 to 20% |
| Grapes pips | 10 to 20% |
| Olive | 20 to 40% |
| Kiwi oil | 1 to 5% |
| Camelina oil | 1 to 10% |
| Acacia fibers | 1 to 10% |
| Selenium | 0 to 1% |
| Zinc | 1 to 5% |
| Essential oil of orange | 0 to 5% |
| Essential oil of lemon | 0 to 5% |

Lycopene is a carotenoid of natural origin of the tomato extract, which has an antioxidant and anti free radical activity larger than vitamin E and beta-carotene. It works on wrinkles and improves the appearance of the skin.

The grapes pips rich in OPC (ProCyanidic oligomer) have also an important antioxidant and anti radical free activity, a further action in the vascular protection and collagen.

The olive tree contains an active ingredient, the oleuropeoside, which has regulatory properties of blood cholesterol and triglyceride, fluidizes the blood and better oxygenates tissues.

Kiwi oil and camelina oil are rich in omega 3, with a chemical composition in polyunsaturated fatty acids very close to the fish oils known for their anti-oxidant, anti free radical and anti-aging properties.

Acacia fibers are soluble vegetable fibers, which facilitate intestinal transit and the use during digestion of micronutrients necessary for the bloom of the skin.

Selenium is part of the structure of the glutathione peroxidase enzyme, which has anti-oxidant and anti-aging properties and is useful in maintaining healthy skin and hair.

Zinc occurs at the level of DNA synthesis and cell regeneration, by activation of the enzyme SOD (SuperOxyde Dismutase), it presents an anti-aging action on skin and phanera.

The essential oil of orange is a cell and skin regenerative.

The essential oil of lemon leads to a beneficial effect on the peripheral micro-circulation.

The Beta-Meditril® potentiates the phytoaromatherapeutic action by its rich phytonutrients.

Such a composition helps to maintain and regulate the structure and brilliance of a younger skin and to counter the effects of aging. Combined with a balanced diet, such a composition brings an active synergy to reduce wrinkles, enhance cell renewal, redensify and harden the skin, slow the impact of time on the face and body, and provide an additional source of omega 3.

### EXAMPLE 2

### Granule A40 HARMONIE FEMININE

We realize a granulation using the Technabio™ process.

The percentages reflect the percentages of active ingredients in the orogranule finished:

| | |
|---|---|
| Beta-Meditril ® | 1 to 10% |
| Yam | 20 to 50% |
| Soya | 20 to 50% |
| Sage | 1 to 10% |
| Fennel | 1 to 10% |
| Liquorice | 1 to 10% |
| Essential oil of verbenone | 1 to 10% |
| Essential oil of orange | 0 to 5% |
| Essential oil of lemon | 0 to 5% |

The yam is rich in diosgenine, natural precursor of progesterone whose deficiency is the cause of menopause disturbances.

The soya is rich in genisteine and daidzeine which are equel precursor which has an important oestrogenic activity.

The sage, which is rich in flavonoids, rosmarinic acid and diterpenes, has an antioxidant, antispasmodic and oestrogenic activity. It works against asthenia and typical night sweating of the pre-menopause and menopause.

The fennel owes its action to its content anetol, which assign a certain natural oestrogen activity.

The liquorice stimulates the immune system, is opposed to the action of free radicals and generates a slight natural oestrogen activity.

The essential oil of orange facilitates the circulation and reduces the heaviness of the legs.

The essential oil of lemon leads to a beneficial effect on the peripheral micro-circulation.

The essential oil of verbenone rosemary has a significant regulatory action of mood, calming, relaxing and unstressful in the nervous system.

Such a composition helps to combat the inconvenience associated with hormonal changes of pre-menopause and menopause. Combined with an appropriate diet, such a composition brings an active synergy to calm the intensity of hot flashes, reduce the impact of irregularities in the menstrual cycle, harmonize mood disorders, decrease the feeling of swelling and outlet weight, and contribute to the alleviation of menopausal symptoms.

### EXAMPLE 3

### Granule A40 SILHOUETTE

We realize a granulation using the Technabio™ process.

The percentages reflect the percentages of active ingredients in the orogranule finished:

| | |
|---|---|
| Beta-Meditril ® | 1 to 10% |
| Green coffee | 10 to 20% |
| Pineapple | 10 to 20% |
| Guarana | 10 to 20% |
| Red grapevine | 10 to 20% |
| Hibiscus | 10 to 20% |
| Essential oil of grapefruit | 0 to 5% |
| Essential oil of orange | 0 to 5% |
| Essential oil of lemon | 0 to 5% |

The green coffee is a general stimulator which has lipolytic and diuretic properties that reduce the inaestheticism associated with cellulite.

The pineapple, rich in bromelaine, powerful proteolytic enzyme, fights effectively against cellulite, reduces oedema. It also has proven action in the detoxification of the body.

The guarana is very rich in caffeine that confers “fat burn” and anti-diuretic properties.

The red grapevine is a venous stimulator, it promotes drainage, and elimination of cellulite.

The hibiscus facilitates the reduction of cellulite and its elimination.

The essential oils of grapefruit and orange promote lipolytic and anti-cellulite action.

The essential oil of lemon leads to a beneficial effect on the peripheral micro-circulation.

Such a composition promotes combustion and destoring of located fat and helps against inaestheticism of cellulite and unsightly bulges. This composition makes an active synergy to strengthen and revitalize the figure, reduce water retention and burn fat, activate the circulation, facilitate the removal of fat, act against overweight, reduce the effects of cellulite and localized unsightly bulges and cancel the sensation of swelling.

### EXAMPLE 4

### Granule A40 DRAINAGE

We realize a granulation using the Technabio™ process.

The percentages reflect the percentages of active ingredients in the orogranule finished:

| | |
|---|---|
| Beta-Meditril ® | 1 to 10% |
| Green tea | 20 to 40% |
| Ash | 10 to 20% |
| Orthosiphon | 10 to 20% |
| Cherry stalk | 10 to 20% |
| Corn | 10 to 20% |
| Essential oil of grapefruit | 0 to 5% |
| Essential oil of orange | 0 to 5% |
| Essential oil of lemon | 0 to 5% |

Green tea is rich in caffeine, polyphenols and flavonoids, has stimulating, antioxidant and detoxifying effects, promoting renal excretion of water.

Ash, whose leaves contain an active anti-inflammatory drug, rutoside, mannitol and potassium salts, facilitates the removal of water and toxins.

The orthosiphon stimulates the elimination function of water, chlorides, uric acid and urea through a double action of vesicular and urinary drainage.

The cherry stalk intensifies the elimination of water and toxins from the body.

Corn and especially corn beard that surrounds the ear, rich in polyphenols, oil and potassium salt, have a specific renal action and presents beneficial virtues for elimination.

The essential oil of grapefruit and orange promote lipolytic and anti-cellulite action.

The essential oil of lemon leads to a beneficial effect on the peripheral micro-circulation.

Such a composition promotes the elimination of excess water and helps to cleanse the body of toxins.

Combined with a slimming diet and adequate physical activity, such a composition brings an active synergy to act against overweight, remove excess water, refine infiltrated tissues, promote toxin elimination, and to promote mass of fat drainage.

For this example, a clinical study was conducted to assess the benefits. The weight gain concerns many women, whatever the causes: hormonal contraception, hormone replacement therapy or dietary aberrations.

It often leads concerned women to take risks through the use of disproportionate treatments, and many women start by resorting to anorectics to get a quick result.

It is well known that the treatment is stopped, they see a resumption of weight. This is the beginning of an escalation with anorectics therapeutic, calming, and miracle regimes.

It is therefore essential to make them aware that it must start with a balanced diet. However, everyone knows that these hypocalorific regimes are often poorly accepted.

Concerned women want a quick result, concerning either the decrease in weight or the sensation of swelling.

It is therefore up to the doctor to exercise pedagogy recalling the dietary rules, but also to take into account the consequences of overweight, including notably the sensation of swelling.

Therefore, we deemed it useful to study the activity of the “Drainage” formula in a female population of 40 to 60 years.

Thus, women volunteers were selected in random order. Some received the “Drainage” formula with a diet program, others only the dietetic program.

The “Drainage” formula consists of a phytonutritional MonoAssociation of green tea, ash, orthosiphon, cherry stalk, corn, spirulina, Beta-Meditril® (extract of olive tree, tomato lycopene, marc grapes, algae complex (fucus, sea lettuce, spirulina), natural aroma, essential oils of grapefruit, lemon and orange, and maltitol.

These active ingredients provide a diuretic, draining and lipolytic synergy.

So far, we do not know any contraindications nor secondary interactions for this composition.

The dosage was determined by the 2 takings a day, not during mealtime, melt in the mouth.

The duration of treatment was 30 days with 3 controls D0, D+15, D+30.

The inclusion criteria were female volunteers with a body mass index (BMI) greater than 24 and less than 30.

The exclusion criteria were taking diuretics or anorectic drugs.

The evaluation criteria are weight, waist, hip and thigh measurements.

Patients also noted the feeling of swelling and bulimic impulses from 0 to 3 depending on their intensity.

| | | |
|---|---|---|
| Number of | | Standard |
| patients | Average | deviation |

<< DRAINAGE >> Formula

| | | | | |
|---|---|---|---|---|
| Age | | 30 | 40.83 | 11.58 |
| Height | | 30 | 167.47 | 5.95 |
| Weight | | 30 | 64.88 | 4.54 |
| Waist | | 30 | 72.93 | 3.71 |
| measurement | | | | |
| Thigh | | 30 | 56.27 | 3.68 |
| measurement | | | | |
| Hip | | 30 | 99.73 | 2.57 |
| measurement | | | | |
| Married | | 23 | — | — |
| Singles | | 6 | — | — |
| Divorced | | 1 | — | — |
| Children | | 22 | — | — |
| Working | | 23 | — | — |
| Without job | | 7 | — | — |
| Tobacco | | 14 | 5.50 | 4.17 |
| addiction/ | | | | |
| day | | | | |
| Alcoholism/ | | 15 | 1.80 | 0.50 |
| day | | | | |
| Systolic | | 30 | 130.83 | 8.08 |
| arterial | | | | |
| tension | | | | |
| Diastolic | | 30 | 76.30 | 4.26 |
| arterial | | | | |
| tension | | | | |
| BMI | 23.00 | 30 | 23.00 | |

DIET only

| | | | | |
|---|---|---|---|---|
| Age | | 30 | 39.60 | 9.99 |
| Height | | 30 | 167.10 | 4.74 |
| Weight | | 30 | 66.47 | 4.06 |
| Waist | | 30 | 73.20 | 5.04 |
| meausrement | | | | |
| Thigh | | 30 | 56.95 | 4.02 |
| measurement | | | | |
| Hip | | 30 | 99.45 | 3.96 |
| measurement | | | | |
| Married | | 16 | — | — |
| Singles | | 12 | — | — |
| Divorced | | 2 | — | — |
| Children | | 17 | — | — |
| Working | | 25 | — | — |
| Without job | | 5 | — | — |
| Tobacco | | 12 | 6.50 | 3.04 |
| addiction/ | | | | |
| day | | | | |
| Alcoholism/ | | 16 | 2.13 | 0.78 |
| day | | | | |
| Systolic | | 30 | 123.43 | 6.54 |
| arterial | | | | |
| tension | | | | |
| Diastolic | | 30 | 80.37 | 3.04 |
| arterial | | | | |
| tension | | | | |
| BMI | 23.67 | | | |

Evolution Formula << Drainage >> D + 15

| | | | |
|---|---|---|---|
| Weight | 30 | 64.16 | 4.53 |
| Waist | 30 | 72.20 | 3.58 |
| measurement | | | |
| Thigh | 30 | 55.80 | 3.59 |
| measurement | | | |
| Hip | 30 | 99.02 | 2.48 |
| measurement | | | |
| Swelling | 24 | 1.80 | 0.40 |
| Hungers | 26 | 2.00 | 0.77 |

Evolution Formula << Drainage >> D + 30

| | | | |
|---|---|---|---|
| Weight | 30 | 63.42 | 4.48 |
| Waist | 30 | 71.67 | 3.57 |
| measurement | | | |
| Thigh | 30 | 55.32 | 3.56 |
| measurement | | | |
| Hip | 30 | 98.45 | 2.47 |
| measurement | | | |
| Swelling | 4 | 1.03 | 0.66 |
| Hungers | 2 | 1.43 | 0.67 |

The benefit of treatment seems obvious. But it should be studied statistically, the patient being his own witness. Remember the principle of the test to determine the significance of differences observed.

Md is the average of differences observed D0−D+15. Sd represents the corresponding standard deviation.

The significance E is determined by the ratio between Md and Sd weighted by the square root of the corresponding number of patients.

The changing parameters is analyzed in the following table:

Evolution and significance of parameters

| | | |
|---|---|---|
| MD | SD | Significance E |

Formula << Drainage >>

| | | | |
|---|---|---|---|
| D0-D15 | | | |
| Weight | 0.72 | 0.32 | 12.00 |
| Waist | 0.73 | 0.33 | 11.00 |
| measurement | | | |
| Thigh | 0.47 | 0.63 | 4.00 |
| measurement | | | |
| Hip | 0.72 | 0.36 | 10.00 |
| measurement | | | |
| Swelling | 0.90 | 0.30 | 16.00 |
| Hungers | 0.47 | 0.56 | 4.00 |
| D0-D30 | | | |
| Weight | 1.47 | 0.45 | 18.00 |
| Waist | 1.27 | 0.40 | 17.00 |
| measurement | | | |
| Thigh | 0.95 | 0.35 | 8.00 |
| measurement | | | |
| Hip | 1.28 | 0.49 | 14.00 |
| measurement | | | |
| Swelling | 1.67 | 0.60 | 15.00 |
| Hungers | 1.03 | 0.75 | 7.00 |

Diet only

| | | | |
|---|---|---|---|
| D0-D15 | | | |
| Weight | 0.53 | 0.52 | 5.00 |
| Waist | 0.62 | 0.48 | 7.00 |
| measurement | | | |
| Thigh | 0.47 | 0.39 | 6.00 |
| measurement | | | |
| Hip | 0.67 | 0.39 | 9.00 |
| measurement | | | |
| Swelling | 0.77 | 0.50 | 8.00 |
| Hungers | 0.77 | 0.67 | 6.00 |
| D0-D30 | | | |
| Weight | 1.37 | 0.82 | 9.00 |
| Waist | 0.62 | 0.48 | 7.00 |
| measurement | | | |
| Thigh | 1.22 | 0.51 | 13.00 |
| measurement | | | |
| Hip | 1.37 | 0.59 | 12.00 |
| measurement | | | |
| Swelling | 1.60 | 0.66 | 13.00 |
| Hungers | 1.13 | 0.72 | 8.00 |

The benefit of treatment was already obvious from the second week. It is felt by the patient and objectified by the measures.

As part of this study, no case of intolerance or neglect was found.

The formula “DRAINAGE” appears to be an appropriate response to the problems resulting from overweight.

### EXAMPLE 5

### Granule A40 CIRCULATION

We realize a granulation using the Technabio™ process.

The percentages reflect the percentages of active ingredients in the orogranule finished:

| | |
|---|---|
| Beta-Meditril ® | 1 to 10% |
| Red grapevine | 20 to 30% |
| Blueberry | 20 to 30% |
| Ginkgo | 10 to 20% |
| Cypress | 10 to 20% |
| Essential oil of orange | 0 to 5% |
| Essential oil of lemon | 0 to 5% |
| Essential oil of cypress | 0 to 5% |
| Essential oil of helichryse | 0 to 5% |

The red grapevine, rich in anthocyanosides, is a venous stimulator, it improves circulation in capillaries, drainage and feeling of light legs.

The blueberry is rich in flavonoids and in anthocyanosides, it facilitates the capillary and lymphatic circulation. It is also working on the vision.

The ginkgo is rich in flavonoids and ginkgolides, it increases the venous tone, improves cerebral irrigation and trap free radicals.

The cypress is rich in flavonolic proanthocyanidols; it protects vessels and activates of the peripheral circulation.

The essential oils of orange and lemon facilitate the circulation.

The essential oil of cypress facilitates the return circulation.

The essential oil of helichryse is anti-inflammatory and slightly anti-coagulation, it facilitates the return circulation.

Such a composition promotes the venous return and the microcirculation for light and reactive legs. With a healthy lifestyle, such a composition brings an active synergy to enable active venous return, facilitating the microcirculation (varicose veins, varicosities, blotches), refine and alleviate heavy legs, soothe hemorrhoids and reduce the effects of unrest menstrual.

### EXAMPLE 6

### Granule A40 TRANSIT

We realize a granulation using the Technabio™ process.

The percentages reflect the percentages of active ingredients in the orogranule finished:

| | |
|---|---|
| Beta-Meditril ® | 1 to 10% |
| Tamarind tree | 20 to 40% |
| Fig | 10 to 30% |
| Plum | 10 to 30% |
| Apple pectin | 10 to 30% |
| Acacia fibers | 5 to 20% |
| Essential oil of orange | 0 to 5% |
| Essential oil of fennel | 0 to 5% |
| Essential oil of rosemary | 0 to 5% |

The tamarind tree is rich in organic acids, salts, pectin and simple sugars which have an osmotic effect causing an increase in the mass of water in the intestinal bolus and stimulating natural evacuation.

The fig, rich in sugars, flavonoids, organic acids and enzymes, has digestive and anti-inflammatory properties, and facilitating the transit.

The plum is well known as a laxative fruit facilitating intestinal transit.

The apple pectin is considered as intestinal antiseptic, useful in the treatment of diarrhea and especially intestinal constipation.

Acacia fibers are soluble dietary fibers useful to stimulate and regulate the transit. It promotes good vitamin absorption in the intestine.

The essential oil of orange is indicated for its calming and sedative abilities.

The essential oil of fennel, rich in phenol, anetol, canfrene, may exercise several activities which the most important are: digestive, antispasmodic, laxative and carminative.

The essential oil of rosemary is recognized for its beneficial properties in the case of liver problems, bad digestion, diarrhea and colitis.

Such a composition is involved in maintaining the physiological flora of the intestine and help to restore daily well-being intestinal. Combined with a balanced diet, such a composition brings an active synergy to better manage constipation, energize lazy intestines, alleviate the difficulties of expulsion, relieve difficult digestion and reduce the frequency of intermittent constipations of travellers.

### EXAMPLE 7

### Granule A40 URINAIRE

We realize a granulation using the Technabio™ process.

The percentages reflect the percentages of active ingredients in the orogranule finished:

| | |
|---|---|
| Beta-Meditril ® | 1 to 10% |
| Heather | 20 to 40% |
| Cranberry | 20 to 40% |
| Cherry stalk | 20 to 40% |
| Blueberry | 1 to 10% |
| Essential oil of orange | 0 to 5% |
| Essential oil of de pine | 0 to 5% |
| Essential oil of lavender | 0 to 5% |
| Essential oil of melaleuca | 0 to 5% |

The heather rich in tannins, acids and arbutin, promotes the elimination of water and has a powerful antiseptic power on the urinary tract.

The cranberry is rich in procyanine and fructose, which give an antibacterial activity helping bacteria to cling to the walls of the urinary epithelium.

The cherry stalk is used for its properties to stimulate the elimination of water, to reduce the frequency of occurrence of urinary problem.

The blueberry, rich in acids and tannins, is used for its general antiseptic properties.

The essential oil of orange is indicated for its calming and sedative abilities.

The essential oil of pine is known for its urinary antiseptic and anti-inflammatory properties.

The essential oil of lavender is considered antiseptic, calming, soothing.

The essential oil of melaleuca is useful for its powerful antiseptic properties especially at the bladder level.

Such a composition promotes naturally a better urinary comfort. Combined with a diet very rich in fibers and liquids and with absolute local hygiene, such a composition brings an active synergy to control small urinary burns, control frequent urination, reduce urinary retention and soothe recurring burns.

### EXAMPLE 8

### Granule A40 DETENTE

We realize a granulation using the Technabio™ process.

The percentages reflect the percentages of active ingredients in the orogranule finished:

| | |
|---|---|
| Beta-Meditril ® | 1 to 10% |
| Passionflower | 10 to 20% |
| Lime tree | 10 to 20% |
| Balm | 10 to 20% |
| Lavender | 10 to 20% |
| Orange tree | 10 to 20% |
| Essential oil of orange flower | 0 to 5% |
| Essential oil of orange | 0 to 5% |

The passionflower contains flavonoids, betacarbolins and maltol responsible for the sedative action. It is recommended for sleep disorders, as well as for anxiety and nervousness.

The lime tree, through its inflorescences is rich in polyphenols and has an action on minor sleep disorders. It calms nervousness and facilitates digestion.

The balm has antispasmodic and digestive properties and sedative activity for the treatment of minor sleep disorders.

The lavender, which is rich in essential oil, has a sedative and anxiolytic activity and also has calming properties and facilitates digestion.

The orange and especially the leaves and flowers provide an aromatic extract with sedative properties against nervousness, irritability and passing sleep disturbances.

The essential oil of orange flowers is sedative and promotes sleep.

The essential oil of orange is indicated for its calming and sedative abilities.

Such a composition promotes better control of daily stress and reparative sleep during the night. Combined with a balanced diet, such a composition provides an active synergy to restore the balance of biological rhythm and daily soothing, ease sleep-inducing, improve the quality of sleep, reduce night-time awakenings, and find an active day after restful sleep.

### EXAMPLE 9

### Granule A40 ARTICULATION

We realize a granulation using the Technabio™ process.

The percentages reflect the percentages of active ingredients in the orogranule finished:

| | |
|---|---|
| Beta-Meditril ® | 1 to 10% |
| Harpagophytum | 20 to 40% |
| Cartilage of shark | 20 to 40% |
| Horsetail | 10 to 20% |
| Nettle | 10 to 20% |
| Meadowsweet | 10 to 20% |
| Essential oil of orange | 0 to 5% |
| Essential oil of lemon | 0 to 5% |
| Essential oil of camomile | 0 to 5% |

The harpagophytum is rich in iridoids, principally harpagoside, which act synergistically by anti-inflammatory and analgesic action in the tendon and joint pains.

The shark cartilage is a natural remineralising rich in chondroitin and glucosamine sulfate.

The horsetail is very rich in mineral elements, including silica, which has a beneficial effect on bone structure, reducing the fragility of the bones and consolidate the fractures.

The nettle, very rich in minerals, vitamins, proteins, provides a beneficial anti-inflammatory activity against rheumatic pain and in the case of fragile bones.

The meadowsweet has a significant anti-inflammatory activity through its salicylates derivatives, which enable it to alleviate pain of minor rheumatism and gout.

The essential oil of orange helps to calm the nervous system.

The essential oil of lemon has a beneficial effect on peripheral micro-circulation.

The essential oil of camomile has calming, balanced, analgesic and anti-inflammatory properties.

Such a composition promotes articular flexibility and improves the cartilage resistance. Combined with a balanced diet, such a composition brings an active synergy to protect and stimulate the responsiveness of joints, relieve painful joints, regain natural mobility and enhance articular cartilage in danger of degradation.

### EXAMPLE 10

### Granule A40 CLA BRULE GRAISSE

We realize a granulation using the Technabio™ process.

The percentages reflect the percentages of active ingredients in the orogranule finished:

| | |
|---|---|
| Beta-Meditril ® | 1 to 10% |
| Conjugated linoleic acid | 1 to 10% |
| Guarana | 20 to 40% |
| Cherry stalk | 20 to 40% |
| Essential oil of grapefruit | 0 to 1% |
| Essential oil of orange | 0 to 1% |

The CLA (Conjugated Linoleic Acid) is an essential fatty acid of the family of omega 3-6, presents in meat and dairy products, allows a rapid burning of fat reserves while reducing the degradation of muscle tissue. The CLA improves the ratio of lean body mass compared to the total fat in the body, reducing the deposit fat, especially at the belly and promoting muscle growth. More than promoting the reduction of weight and fat in the body, the CLA increases the natural defences, supports a healthy response to inflammation and is an effective provider of antioxidant protection. Considering that our bodies do not produce CLA, it must be added to our daily diet as a nutritional supplement.

The guarana, very rich in caffeine, increases the basic metabolism of cells by activating the combustion of fats and stimulating the release of catecholamines, adrenaline, which allow the body to burn fat faster. The guarana has tonic and stimulating properties.

The cherry stalk stimulates the elimination of water and toxins from the body.

The essential oil of grapefruit and orange facilitate lipolytic and anti-cellulitis action.

Such a composition promotes combustion and destoring of localized fat, facilitates their transformation into lean body mass and their elimination. Combined with a correct physical and food hygiene, such a composition brings an active synergy to burn fat, transform fat into muscle mass, strengthen and revitalize the skyline, drain by activating the urinary elimination of water, toxins and fats, and act on the overweight.

### EXAMPLE 11

### Granule Z24 CEREBRAL

We realize a granulation using the Technabio™ process.

The percentages reflect the percentages of active ingredients in the orogranule finished:

| | |
|---|---|
| Beta-Meditril ® | 1 to 10% |
| Ginkgo | 20 to 40% |
| Hawthorn | 20 to 40% |
| Olive tree | 10 to 30% |
| Basil | 10 to 30% |
| Essential oil of orange | 0 to 5% |
| Essential oil of lemon | 0 to 5% |
| Essential oil of basil | 0 to 5% |

The ginkgo, which is rich in flavonoids, facilitates the capillary circulation, particularly in the brain, facilitating the prevention of circulatory problems and stimulating the threshold of attention, memory and reaction time.

The hawthorn facilitates blood circulation, helps to regulate heartbeat and has soothing and calming properties on nervousness and anxiety.

The olive tree, whose leaves are rich in oleuropeoside, helps to protect blood pressure by lowering blood circulating cholesterol and triglycerides. Its diuretic action harmoniously completes these beneficial actions.

The basil, very rich in essential oil and tannins, has antispasmodic properties, particularly in the digestive tract, and tonic properties in the nervous system, and may also enhance the immune system.

The essential oil of orange facilitates circulation.

The essential oil of lemon leads to a beneficial effect on the peripheral micro-circulation.

The essential oil of basil is tonic of the nervous system and stimulates digestion.

Such a composition promotes the improvement of brain function, boosts cerebral micro-circulation and stimulates the vascular protection. Combined with an adapted diet, such a composition brings an active synergy to promote cerebral irrigation, stimulate cerebral performance, memory and concentration, reduce the effects of senescence disorders, contribute to the reduction of the atheromatous plaque and participate in the regulation of blood pressure.

### EXAMPLE 12

### Granule Z24 TONIC H

We realize a granulation using the Technabio™ process.

The percentages reflect the percentages of active ingredients in the orogranule finished:

| | |
|---|---|
| Beta-Meditril ® | 1 to 10% |
| Ginseng | 20 to 40% |
| Ginger | 20 to 40% |
| Eleutherocoque | 10 to 30% |
| Spirulina | 10 to 30% |
| Essential oil of ginger | 0 to 5% |
| Essential oil of mint | 0 to 5% |
| Essential oil of orange | 0 to 5% |

The ginseng is rich in ginsenosides, which confer tonic physical and intellectual properties. It improves the resistance to the effort and is considered by Chinese medicine as an aphrodisiac.

The ginger has a remarkable effect on the gastric mucosa, reducing nausea and vomiting, and is considered as a stimulator of the nervous system with tonic physical, intellectual, and sexual properties.

The eleutherocoque, also known as Siberian ginseng, is used for its adaptogenic properties and ability to resist to stress. It is a stimulator of the central nervous system and the adrenal and seminal glands.

The spirulina is a freshwater alga very rich in micronutrients (trace elements, vitamins), phenylalanine and tyrosine acting effectively in states of physical and sexual fatigue.

The essential oil of ginger is a physical, intellectual, sexual tonic stimulator and facilitates digestion.

The essential oil of mint is invigorating and soothing at the same time.

The essential oil of orange helps to calm the anxiety to do well.

Such a composition promotes physical and sexual vitality, increases resistance to fatigue and improves endurance. Such a composition brings an active synergy by stimulating physical and intellectual performance, optimizing resistance to fatigue, harmonizing fall in passing shape, increasing libido and controlling sexual stress.

### EXAMPLE 13

### Granule Z24 DEFENSES NATURELLES

We realize a granulation using the Technabio™ process.

The percentages reflect the percentages of active ingredients in the orogranule finished:

| | |
|---|---|
| Beta-Meditril ® | 1 to 10% |
| Shiitake | 20 to 40% |
| Pine | 20 to 40% |
| Propolis | 20 to 40% |
| Grapefruit pip | 10 to 30% |
| Essential oil of orange | 0 to 5% |
| Essential oil of pine | 0 to 5% |
| Essential oil of eucalyptus | 0 to 5% |

The shiitake, fungus widely used in the Asian diet, is rich in amino acids, vitamin C, minerals and polysaccharides that confer its immune regulator properties. It increases the action of natural killer cells, and also has an antiviral, antibacterial and antiparasitic activity.

The pine, and in particular its buds, have anti-tussive and expectorant properties. It favours decongestion of the upper respiratory tract.

The propolis, resinous glue lining the hives, is rich in vitamins and flavonoids. Bactericidal agent and immunostimulator by the production of antibodies, it is also anti-pain agent, nervous calming, anti-neurasthenic, and bio stimulating in terms of blood circulation.

The grapefruit pip is rich in bioflavonoids and vitamin C. The extract of grapefruit pips is not only effective in eliminating viruses and bacteria, but also in that of fungi and parasites.

The essential oil of orange is a general antiseptic.

The essential oil of pine acts as a general antiseptic and specific of high upper tracts.

The essential oil of eucalyptus is specific for lower respiratory tracts, decongestant and expectorant.

Such a composition promotes the protection of the immune system and contributes to a better resistance to aggression. Combined with a balanced diet, such a composition brings an active synergy to stimulate natural responsiveness of the body, increase the resistance to aggression, reduce the impact of recurrences, and improve tolerance to seasonal changes.

### EXAMPLE 14

### Granule Z24 BLANCHIDENT

We realize a granulation using the Technabio™ process.

The percentages reflect the percentages of active ingredients in the orogranule finished:

| | |
|---|---|
| Beta-Meditril ® | 1 to 10% |
| Sage | 10 to 30% |
| Lemon juice | 10 to 30% |
| Soda bicarbonate | 1 to 10% |
| Maltitol | 40 to 60% |
| Calcium Glycerophosphate | 1 to 10% |
| Essential oil of mint | 0 to 5% |

The sage, which is rich in flavonoids, rosmarinic acid and di-terpene, has antioxidant activities which enable it to act on the formation of dental plaque.

The lemon juice is deemed to restore whiteness to tooth enamel.

The soda bicarbonate neutralizes the acid attacks due to food responsible for tooth aggression and tooth decays. The soda bicarbonate in local application on the teeth helps to restore teeth whiteness.

The maltitol is a polyol, sweetener, acariogene, which protects against acid attacks of bacteria.

The calcium glycerophosphate, thanks to its rich calcium immediately available, helps to strengthen tooth enamel.

The essential oil of mint is a general antiseptic, which helps to reduce oral microbial flora, perfumes breath and facilitates digestion.

Such a composition increases the resistance of teeth to attacks of time and smoke, and protects from acid attacks of food. Such a composition brings an active synergy to protect and reduce the formation of dental plaque, neutralize acid food attacks, protect and strengthen tooth enamel, help to restore the teeth whiteness, reduce the impact of tobacco on breath and colour teeth, and promote the purification of breath.

### EXAMPLE 15

### Granule Z24 NOTOBACKO

We realize a granulation using the Technabio™ process.

The percentages reflect the percentages of active ingredients in the orogranule finished:

| | |
|---|---|
| Beta-Meditril ® | 1 to 10% |
| Kudzu | 20 to 40% |
| Marshmallow | 10 to 30% |
| Blackcurrant | 10 to 30% |
| Plantain | 10 to 30% |
| Essential oil of mint | 0 to 5% |

The kudzu contains many ingredients that exert a protective action of the liver and antioxidant. It has an activity on the metabolism of alcohol and drugs, such as tobacco. Its anti-stress action makes coming off easier.

The marshmallow by its mucilages, moisturizes, softens and soothes the irritated buccal and respiratory mucosa, particularly by smoke. Its polysaccharides confers an immunostimulating effect.

Blackcurrants are rich in sugars and organic acids which have a calming and soothing action at the mouth.

The plantain alters the taste of cigarettes, making it less enjoyable for the smoker.

The essential oil of mint is antiseptic of the buccal mucosa and purify the breath.

Such a composition promotes the reduction in the urge to smoke and the withdrawal feeling, and reduces nervousness and the sensation of hunger. Such a composition brings an active synergy to strengthen the will to stop smoking, reduce stress related to smoking coming off, soften and calm the irritability of the buccal mucosa and throat due to the smoke, calm bulimic urges related to smoking cessation and complement the actions of methods for coming off.

Thus, the invention must be understood in a broad sense to encompass methods or variations of preparation of granules that are not specifically described in this description, but which would be equivalent to the methods described or that can be deducted from characteristics of the invention by routine operations carried out by a person skilled in the art.

In particular, the manufacture of granules according to the invention can be carried out in a fluidized bed or using any other devices or facilities able to produce granules.

1. A composition comprising a nutrient and a phytotherapeutic compound, wherein said composition further comprises an essential oil and/or essence and is in the form of a granule.

2. The composition of claim 1, wherein the nutrient is a protein, a carbohydrate, a lipid, a fiber, an antioxidant, an anti free radical, a vitamin, a mineral salt, a trace element, or a mixture of those.

3. The composition of claim 1, wherein the nutrient concentration is comprised between approximately 1 and approximately 50% of the total weight of the composition.

4. The composition of claim 1, wherein the phytotherapeutic compound is an active plant extract of a whole plant or a plant part.

5. The composition of claim 1, wherein the phytotherapeutic compound concentration is comprised between approximately 1 and approximately 80% of the total weight of the composition.

6. The composition of claim 1, wherein the essential oil and/or essence is obtained by distillation drive to water vapor of an aromatic plant or by pressing and scraping citrus fruit zests.

7. The composition of claim 1, wherein the concentration of essential oil and/or essence is comprised between approximately 0 and approximately 50% of the total weight of the composition.

8. The composition of claim 1, wherein the granule is orally soluble.

9. The composition of claim 1, wherein the nutrient is a nutritional complex comprising at least:
lycopene,
at least one of tannin or polyphenol,
an oleuropeoside,
fucus,
lettuce Seas, and
spirulina .

10. A process for preparing a composition in the form of a granule according to claim 1, wherein it comprises the following steps:
granulation in the presence of a crystalline solid powdery phase comprising a nutrient and a phytotherapeutic compound and a liquid phase, which comprises a gum and an essential oil and/or essence;
coating by spraying of the liquid phase and sprinkling of solid crystalline powdery phase;
drying the coated granules obtained; and
screening for obtaining calibrated granules.

11. A process according to claim 1 wherein all compounds are incorporated in dry form.

12. The composition of claim 1 comprising a nutrient, a phytotherapeutic compound and an essential oil and/or essence in a granule form, wherein said composition has a synergistic phytotherapeutic, aromatherapeutic and nutritherapeutic activity.

13. The composition of claim 1 comprising a nutrient, a phytotherapeutic compound and an essential oil and/or essence in form of a granule, wherein said composition is used as a complement of a varied and balanced diet for indications such as aging, weight loss, discomfort related to hormonal changes of the pre-menopause and menopause, drainage, digestion, intestinal transit, relaxation, calm, microcirculation, appeasement of the bacterial flora, painful joints, physical and mental fatigue, protection of the immune system, or even whiten teeth and reduce urge to smoke.
